(19) United States
(12) Patent Application Publication
(10) Pub. No.: US 2017/0319260 A1
(43) Pub. Date: Nov. 9, 2017

(54) COORDINATION/CONTROL OF MULTIPLE MEDICAL DEVICES AT A SITE

(71) Applicant: Analog Devices, Inc., Norwood, MA (US)

(72) Inventors: Hung D. Ngo, Waltham, MA (US); Christopher W. Hyde, Hollis, NH (US); Michael C. W. Coln, Lexington, MA (US); Lalinda D. Fernando, Westford, MA (US)

(21) Appl. No.: 15/587,622

(22) Filed: May 5, 2017

Related U.S. Application Data
(60) Provisional application No. 62/332,076, filed on May 5, 2016.

(51) Int. Cl. A61B 18/12; A61B 18/14; A61B 18/00

(52) U.S. Cl. CPC A61B 18/1206; A61B 18/1492; A61B 2018/00351; A61B 2018/00708; A61B 2018/00577

(57) ABSTRACT

According to one configuration, a controller controls use of multiple medical devices at a medical site. For example, a first medical device at the medical site is operable to perform one or more tissue ablation operations; a second medical device at the medical site is operable to perform non-ablation operations (such as tissue monitoring operations). The controller implements a control sequence to control switching between different operational modes including a first mode and a second mode. The first mode and corresponding first windows of time enable the first medical device to perform an ablation operation; the second mode and corresponding windows of time disable the first medical device from performing the ablation operation so that the second medical device operates without interference from the first medical device performing the ablation operation.

### RELATED APPLICATIONS

This application is related to and claims the benefit of earlier filed U.S. Provisional Patent Application Ser. No. 62/332,076 entitled “SYSTEMS AND METHODS OF MONITORING INTRA-CARDIAC SIGNALS DURING MAPPING AND/OR ABLATION OF CARDIAC TISSUE,” Attorney Docket No. ADI16-03(ACQ402-1-US)P, filed on May 5, 2016, the entire teachings of which are incorporated herein by this reference.

### FIELD OF THE DISCLOSURE

The present disclosure is generally directed to apparatus, methods, computer-readable media, etc., to coordinate/control multiple medical devices to perform operations, tasks, functions, etc., at a medical site. Certain specific embodiments herein are directed to electrophysiological (EP) catheter systems and methods, and more specifically to EP catheter systems and methods for use in performing tasks such as mapping, monitoring, ablation of cardiac tissue, etc.

### BACKGROUND

Cardiac mapping and ablation systems are known for treating cardiac arrhythmias in human subjects. Such cardiac arrhythmias can result from improper electrical activity of a human subject's heart, and can include an irregular heartbeat that is either too fast (also known as “tachycardia arrhythmia”) or too slow (also known as “bradycardia arrhythmia”).

While some types of cardiac arrhythmias may not be life threatening, other types of cardiac arrhythmias may ultimately become sufficiently serious to cause stroke, heart failure, or cardiac arrest. Accurate diagnosis and treatment of cardiac arrhythmias can therefore be vital for reducing the risk of disability and for saving lives.

A conventional cardiac mapping and ablation system typically includes a cardiac mapping/ablation catheter, a radio frequency (RF) ablation energy source, one or more skin surface electrodes, a cardiac mapping/signal monitor, and a stimulation/pacing signal generator. The cardiac mapping/ablation catheter can include, at its distal end, a plurality of intra-cardiac electrodes and at least one ablation electrode, which is electrically connectable to the RF ablation energy source.

In a typical mode of operation, the distal end of the catheter is positioned in the heart of a human subject in order to locate one or more sites within the heart that are associated with tachycardia or bradycardia arrhythmia. To that end, the cardiac mapping and ablation system maps electrical activation sequences of the human subject's heart using the intra-cardiac electrodes and the skin surface electrodes, which can be affixed to the skin of the human subject's body using adhesive patches.

Subsequent to collecting data, the system then displays, on the cardiac mapping/signal monitor, one or more resulting cardiac mapping images indicating the locations of the arrhythmogenic sites within the human subject's heart. Having located the arrhythmogenic sites, the distal end of the catheter is manipulated and guided within the human subject's heart until the ablation electrode is proximate to each arrhythmogenic site, thereby allowing the ablation electrode to ablate cardiac tissue at the arrhythmogenic site location by an application of RF ablation energy from the RF ablation energy source.

While the ablation electrode ablates the cardiac tissue at the respective arrhythmogenic site locations, one or more stimulation/pacing signals generated by the stimulation/pacing signal generator can be applied to the human subject's heart, as needed.

Further, one or more intra-cardiac signals (e.g., intra-cardiac electrogram (IEGM) signals, tissue impedance (TI) signals) can be measured using the intra-cardiac electrodes, and the intra-cardiac signal measurements can be monitored using the cardiac mapping/signal monitor.

### SUMMARY OF THE DISCLOSURE

This disclosure includes the observation that certain types of medical procedures interfere with each other, requiring the procedures to be performed at substantially different times. For example, a first medical device can be used to perform ablation of heart tissue the application of RF (Radio Frequency) energy. A second medical device may be used to monitor attributes of the heart tissue as previously discussed. Presence of the RF energy from the first medical device may interfere with use of the second medical device to perform an operation such as monitoring attributes of the heart tissue. Accordingly, certain conventional medical systems require completion of an ablation operation using a first medical device before using the second medical device to monitor tissue at a medical site where the ablation was performed.

According to one embodiment, a controller as described herein coordinates use of multiple medical devices at a medical site. Assume that a medical system includes a first medical device and a second medical device. The first medical device is operated to perform a first medical operation such as ablation at the medical site; the second medical device is operated to perform a second medical operation such as one or more non-ablation operations at the medical site. To coordinate use of the first medical device and the second medical device, the controller implements a control sequence to control switching between different operational modes including a first mode (such as an ablation mode) and a second mode (such as a non-ablation mode).

In one embodiment, during windows of operating in the first mode, the controller enables the first medical device to perform a first operation such as an ablation operation; during windows of time operating in the second mode, the controller disables the first medical device from performing the ablation operation. Because the first medical device is disabled from performing the ablation operation during windows of operating in the second mode, the second medical device is able to perform functions without interference from a respective ablation operation. Accordingly, in one embodiment, the second medical device can be used to perform one or more non-ablation operations during windows of time in which the first medical device is disabled from performing the ablation operation.

If desired, the second medical device is optionally prevented from performing non-ablation operations (for example, tissue monitoring operations) during the first mode.

In accordance with further embodiments, if desired, the feedback (such as monitor data) from the second medical device may be ignored during windows of time in which the medical system operates in the first mode (ablation mode).

In accordance with further embodiments, the first medical device delivers a respective sequence of energy pulses during each window of time operating in the first mode such that the first medical device (such as a first catheter device) delivers discontinuous delivery of multiple windows of energy pulses to the medical site to perform a single ablation operation. In other words, during a first window of time in which the medical system operates in the first mode, the first medical device starts an operation of ablating particular tissue at the medical site; during a second window of time in which the medical system operates in the second mode, the first medical device discontinues (pauses) the operation of ablating particular tissue at the medical site; during a third window of time in which the medical system operates in the first mode again, the first medical device continues the operation of ablating the particular tissue at the medical site; during a fourth window of time in which the medical system operates in the second mode again, the first medical device discontinues the operation of ablating the particular tissue at the medical site; during a fifth window of time in which the medical system operates in the first mode again, the first medical device continues the operation of ablating the particular tissue at the medical site; and so on.

Note that a magnitude of the energy pulses is sufficiently high such that the discontinuous delivery of multiple windows of energy pulses provides the same ablation results as if the first medical device were otherwise operated in a continuous mode to ablate the tissue at medical site (such as in the chamber of a heart or other site). In other words, windows of operating in the second mode (non-ablation mode) are sufficiently short in time that the corresponding tissue being ablated does not have time to substantially cool, resulting in ablation similar to a continuous mode.

In one embodiment, because the amount of energy supplied by the first medical device is elevated to account for operating in the discontinuous ablation mode, the first medical device is configured to supply a sufficiently high amount to account for the OFF time an appropriate amount of energy per unit time to perform the ablation operation.

In accordance with further embodiments, the windows of time of operating in the second mode are so short in time that the thermal decay (drop in temperature) of tissue being ablated during windows of time operating in the second mode (non-ablation times) is insignificant with respect to the ablation process.

The second medical device is optionally a multi-sensor device including multiple sensors. Each of the sensors monitors respective tissue at a different location of the medical site in which the first medical device performs ablation. In one embodiment, the multi-sensor device is activated to collect data during windows of time of operating in the second mode (non-ablation windows).

In certain instances, it is desirable to display monitored attributes of the medical site (as collected from the second medical device) on a display screen. To this end, the system can include a display screen operable to display detected attributes of the different locations of the medical site as sensed by the multiple sensors. In one embodiment, the medical system displays the attributes of the different locations as detected by the multi-sensor device during windows a time of operating in the second mode (non-ablation windows), which occur in between windows of time operating in the first mode (ablation windows).

Further embodiments herein include a signal generator resource to produce ablation pulses (pulses of RF energy) to ablate the tissue at the medical site using the first medical device. The first medical device applies a respective set of multiple ablation pulses generated by the generator resource during each of multiple windows of time of operating in the first mode. As previously discussed, the controller spaces apart the windows allocated to operate in the first mode via windows allocated to operate in the second mode (via quiescent times) such that the first medical device is disabled from applying continuous pulses of energy to the tissue at the medical site.

The controller as discussed herein can be configured to receive a command from a user operating the first medical device to deliver energy pulses to the medical site. In response to receiving the command: i) during a first window in which the control sequence indicates to operate in the first mode, the first medical device (such as a first catheter device) delivers a first sequence of energy pulses through the first medical device to target tissue at the medical site to perform an ablation operation; ii) during a second window in which the control sequence indicates to operate in the second mode, the first medical device discontinues delivering any energy pulses through the first medical device to the target tissue at the medical site; iii) during a third window in which the control sequence indicates to operate in the first mode again, the first medical device resumes delivery of a second sequence of energy pulses through the first medical device to the target tissue at the medical site; iv) during a fourth window in which the control sequence indicates to operate in the second mode again, the first medical device discontinues delivering of any energy pulses through the first medical device to the target tissue at the medical site; and so on.

As previously discussed, during windows of time in which the control sequence indicates to operate in the second mode, the second medical device can be operated to perform a function such as monitoring the tissue at the medical site. If desired, the second medical device can be operated during windows of time of operating in the first mode in which the first medical device delivers the sequence of RF energy pulses to the tissue of medical site.

Operation of the first medical device during the first mode may or may not interfere with the second medical device monitoring the tissue and providing feedback regarding the results of monitoring. Further embodiments herein optionally include a filter resource to filter feedback received from the second medical device during windows of time in which the control sequence indicates to operate in the first mode. In other words, the control sequence can be used to identify windows of time in which the system operates in the first mode. If it is known that the first medical device delivers one or more energy pulses during the first mode, the filter resource provides a way of identifying such windows of time, which may result in interference with respect to the second medical device collecting data during such times. Feedback in such windows of time of operating in the first mode can be disregarded. Accordingly, it may not be necessary to prevent the second medical device from performing operations during windows of time of operating in the first mode because such data can be ignored.

As another possible safeguard against the ill effects of the first medical device outputting the energy pulses during the first mode, embodiments herein can include delaying activation of the second medical device with respect to a time of transitioning from operating in the first mode to operating in the second mode (of monitoring and collecting measurement data) such that the affects of the RF energy (used to perform ablation at the medical site) is given time to dissipate before using feedback from the second medical device again.

In accordance with yet further embodiments, each respective set of ablation pulses can be configured to include an integer number of energy pulses (integer number of complete periods or cycles) to deliver to the tissue at the medical site while reducing generation of RF (Radio Frequency) noise at the medical site. In other words, embodiments herein can include precisely controlling attributes (start and stop times, phase angles, number of pulses, amplitude, etc.) of the energy pulses used to perform the ablation operation in order to reduce the amount of noise at the medical site.

In accordance with further specific embodiments, improved electrophysiological (EP) catheter systems and methods are disclosed for use in performing mapping and/or ablation of cardiac tissue. For example, the first medical device as discussed herein can be a first catheter (tissue ablation catheter) used to perform ablation of cardiac tissue. The second medical device can be a second catheter (such as a cardiac monitor catheter) used to monitor attributes of the cardiac tissue.

If desired, the first medical device and the second medical device can be configured in a same catheter device to perform ablation and monitoring functions spaced apart via windows as discussed herein.

The disclosed EP catheter systems and methods employ a cardiac monitor catheter that includes an elongated catheter body having a proximal end and a distal end, a control handle disposed at the proximal end of the catheter body, and an electrode assembly disposed at the distal end of the catheter body. The electrode assembly of the cardiac monitor catheter includes a plurality of intra-cardiac electrodes, which can be employed to map electrical activation sequences of the heart of a human subject for locating one or more arrhythmogenic sites within the human subject's heart.

The tissue ablation catheter delivers RF ablation energy from a RF (Radio Frequency) ablation source to the cardiac tissue at the arrhythmogenic site locations. The plurality of intra-cardiac electrodes of the cardiac monitor catheter can be further employed to monitor measurements of intra-cardiac signals (e.g., intra-cardiac electrogram (IEGM) signals, tissue impedance (TI) signals) while the cardiac tissue is being ablated at the respective arrhythmogenic site locations via the ablation catheter.

The cardiac catheter further optionally includes one or more integrated circuits disposed within the catheter body toward its distal end. The respective integrated circuits are operable to transmit cardiac mapping/navigation signals to the plurality of intra-cardiac electrodes for mapping of the heart's electrical activation sequences, as well as receive the intra-cardiac signal measurements from the plurality of intra-cardiac electrodes during the ablation of the cardiac tissue. Via the ablation catheter, the RF ablation energy are applied to the cardiac tissue at the arrhythmogenic site locations in a series of high amplitude RF ablation pulses during each of one or more ablation intervals (such as windows of time operating in the ablation mode); the plurality of intra-cardiac electrodes of the cardiac monitor catheter monitor the low amplitude intra-cardiac signal measurements during one or more quiescent intervals (such as windows of time operating in the non-ablation mode) between the respective RF ablation pulse windows (such as windows of time operating in the ablation mode).

In certain further embodiments, a method of performing cardiac ablation in the heart of a human subject includes positioning the cardiac catheter at the medical site (such as a human subject's heart), and generating, by a radio frequency (RF) ablation signal generator, a series of high amplitude RF ablation pulses during each of one or more predetermined ablation intervals. The method further includes applying, by the ablation catheter, one or more series of the high amplitude RF ablation pulses to cardiac tissue of the heart. The method still further includes monitoring, by a cardiac signal monitor such as a cardiac monitor catheter, one or more low amplitude intra-cardiac signal measurements during one or more quiescent intervals between two or more of the high amplitude RF ablation pulses generated during one or more of the predetermined ablation intervals, and displaying, on a display of the cardiac signal monitor, one or more waveforms corresponding to the low amplitude intra-cardiac signal measurements.

By providing a cardiac catheter that includes, toward a distal end of the catheter body, one or more integrated circuits for transmitting cardiac mapping/navigation signals and receiving intra-cardiac signal measurements, the amount of catheter cabling required to access and monitor such intra-cardiac signal measurements from a multitude of intra-cardiac electrodes can be minimized. Moreover, by applying RF ablation energy through one or more ablation electrodes in a series of RF ablation pulses, and monitoring such intra-cardiac signal measurements during quiescent intervals between the respective RF ablation pulses, unwanted interference between high amplitude RF ablation signals and low amplitude intra-cardiac signals can be reduced and/or eliminated without the need for large and costly filter components.

These and other more specific embodiments are disclosed in more detail below.

Note that any of the resources as discussed herein can include one or more computerized devices, medical devices, mobile devices, servers, base stations, wireless playback equipment, handheld or laptop computers, or the like to carry out and/or support any or all of the method operations disclosed herein. In other words, one or more computerized devices or processors can be programmed and/or configured to operate as explained herein to carry out the different embodiments as described herein.

Yet other embodiments herein include software programs to perform the steps and operations summarized above and disclosed in detail below. One such embodiment comprises a computer program product including a non-transitory computer-readable storage medium (i.e., any computer readable hardware storage medium or hardware storage media disparately or co-located) on which software instructions are encoded for subsequent execution. The instructions, when executed in a computerized device (hardware) having a processor, program and/or cause the processor (hardware) to perform any of the operations disclosed herein. Such arrangements are typically provided as software, code, instructions, and/or other data (e.g., data structures) arranged or encoded on a non-transitory computer readable storage media such as an optical medium (e.g., CD-ROM), floppy disk, hard disk, memory stick, memory device, etc., or other a medium such as firmware in one or more ROM, RAM, PROM, etc., and/or as an Application Specific Integrated Circuit (ASIC), etc. The software or firmware or other such configurations can be installed onto a computerized device to cause the computerized device to perform any operations explained herein.

Accordingly, embodiments herein are directed to methods, apparatuses, computer program products, computer-readable media, etc., that support operations as discussed herein.

One embodiment includes a computer readable storage media and/or a apparatus having instructions stored thereon to facilitate monitoring and ablation of bio-media. For example, in one embodiment, the instructions, when executed by computer processor hardware, cause the computer processor hardware (such as one or more processor devices) to: implement a control sequence that switches between different operational modes over time, the different operational modes including a first mode and a second mode; enable a first medical device to output an energy signal during windows of time of operating in the first mode to perform a medical operation at the medical site; and disable the first medical device from outputting the energy signal during windows of time of operating in the second mode.

The ordering of the steps above has been added for clarity sake. Note that any of the processing steps as discussed herein can be performed in any suitable order.

Other embodiments of the present disclosure include software programs and/or respective hardware to perform any of the method embodiment steps and operations summarized above and disclosed in detail below.

It is to be understood that the apparatus, method, system, instructions on computer readable storage media, etc., as discussed herein also can be embodied strictly as a software program, firmware, as a hybrid of software, hardware and/or firmware, or as hardware alone such as within a processor (hardware or software), or within an operating apparatus or a within a software application.

As discussed herein, techniques herein are well suited for use in the field of bio-media monitoring and tissue ablation applications. However, it should be noted that embodiments herein are not limited to use in such applications and that the techniques discussed herein are well suited for other applications as well.

Additionally, note that although each of the different features, techniques, configurations, etc., herein may be discussed in different places of this disclosure, it is intended, where suitable, that each of the concepts can optionally be executed independently of each other or in combination with each other. Accordingly, the one or more present inventions as described herein can be embodied and viewed in many different ways.

Also, note that this preliminary discussion of embodiments herein purposefully does not specify every embodiment and/or incrementally novel aspect of the present disclosure or claimed invention(s). Instead, this brief description only presents general embodiments and corresponding points of novelty over conventional techniques. For additional details and/or possible perspectives (permutations) of the invention(s), the reader is directed to the Detailed Description section and corresponding figures of the present disclosure as further discussed below.

### BRIEF DESCRIPTION OF THE DRAWINGS

FIG. 1 is an example diagram illustrating a medical system and patient according to embodiments herein.

FIG. 2 is an example diagram illustrating use of a first medical device and a second medical device used at a medical site according to embodiments herein.

FIG. 3 is an example diagram illustrating a medical system according to embodiments herein.

FIG. 4 is an example diagram of a plurality of circuits to monitor a medical site according to embodiments herein.

FIG. 5 is an example diagram illustrating circuitry according to embodiments herein.

FIG. 6 is a graph illustrating use of different frequencies in a frequency spectrum to carry out medical tasks according to embodiments herein.

FIG. 7 is an example diagram illustrating windows of operating in different modes according to embodiments herein.

FIG. 8 is an example diagram illustrating operation of a first medical device and a second medical device during different windows of time according to embodiments herein.

FIG. 9 is an example diagram illustrating a method according to embodiments herein.

FIG. 10 is an example diagram illustrating a computer architecture in which to execute one or more applications according to embodiments herein.

FIGS. 11-12 are example diagrams illustrating various methods according to embodiments herein.

The foregoing and other objects, features, and advantages of the embodiments herein will be apparent from the following more particular description, as illustrated in the accompanying drawings in which like reference characters refer to the same parts throughout the different views. The drawings are not necessarily to scale, with emphasis instead being placed upon illustrating the embodiments, principles, concepts, etc.

### DETAILED DESCRIPTION

Embodiments herein include improved electrophysiological (EP) catheter system and methods for use in mapping and/or ablating of cardiac tissue.

In one embodiment, the disclosed EP catheter systems and methods employ a cardiac catheter that includes an elongated catheter body having a proximal end and a distal end, a control handle disposed at the proximal end of the catheter body, and an electrode assembly disposed at the distal end of the catheter body.

The electrode assembly includes a plurality of intra-cardiac electrodes, which is employed to map electrical activation sequences of the heart of a human subject for locating one or more arrhythmogenic sites within the human subject's heart. The cardiac tissue is ablated at the arrhythmogenic site locations via application of RF ablation energy from a radio frequency (RF) ablation energy source. The plurality of intra-cardiac electrodes is further employed to monitor measurements of one or more intra-cardiac signals (e.g., intra-cardiac electrogram (IEGM) signals, tissue impedance (TI) signals) while the cardiac tissue is being ablated at the respective arrhythmogenic site locations.

The disclosed EP catheter systems and methods as discussed herein avoid at least some of the drawbacks of conventional EP catheter systems and methods, which often require significant cabling to access and monitor intra-cardiac signal measurements from a plurality of intra-cardiac electrodes, as well as large and costly filter components to remove unwanted interference between high amplitude RF ablation signals and low amplitude intra-cardiac signals.

To minimize the need for such cabling, the cardiac catheter further includes one or more integrated circuits disposed within the catheter body toward its distal end. The respective integrated circuits are operable to: (1) receive cardiac mapping/navigation signals from a cardiac mapping/signal monitor, (2) convert the cardiac mapping/navigation signals from digital form to analog form, (3) transmit the cardiac mapping/navigation signals in analog form to the plurality of intra-cardiac electrodes for use in mapping the heart's electrical activation sequences, (4) receive intra-cardiac signal measurements from the plurality of intra-cardiac electrodes during or in between windows of ablation of the cardiac tissue, (5) convert the intra-cardiac signal measurements from analog form to digital form, and (6) transmit the intra-cardiac signal measurements in digital form to the cardiac mapping/signal monitor.

In this way, the transmission of such intra-cardiac signal measurements from a multitude of intra-cardiac electrodes to the cardiac mapping/signal monitor can be facilitated, while reducing cabling requirements from the distal end of the catheter body to its proximal end.

Moreover, to obviate the need for such large and costly filter components, the RF ablation energy is applied to the cardiac tissue at the arrhythmogenic site locations in a series of high amplitude RF ablation pulses during each of one or more ablation intervals (windows of time). Further, the plurality of intra-cardiac electrodes monitor the intra-cardiac signal measurements during one or more quiescent intervals (windows of time) between the respective RF ablation pulses.

As further described herein, by applying the RF ablation energy in a series of RF ablation pulses during the ablation intervals, and monitoring the intra-cardiac signal measurements during the quiescent intervals between the respective RF ablation pulses as discussed herein, RF ablation signal interference (which can be superimposed onto incoming intra-cardiac signals) are essentially eliminated without filtering.

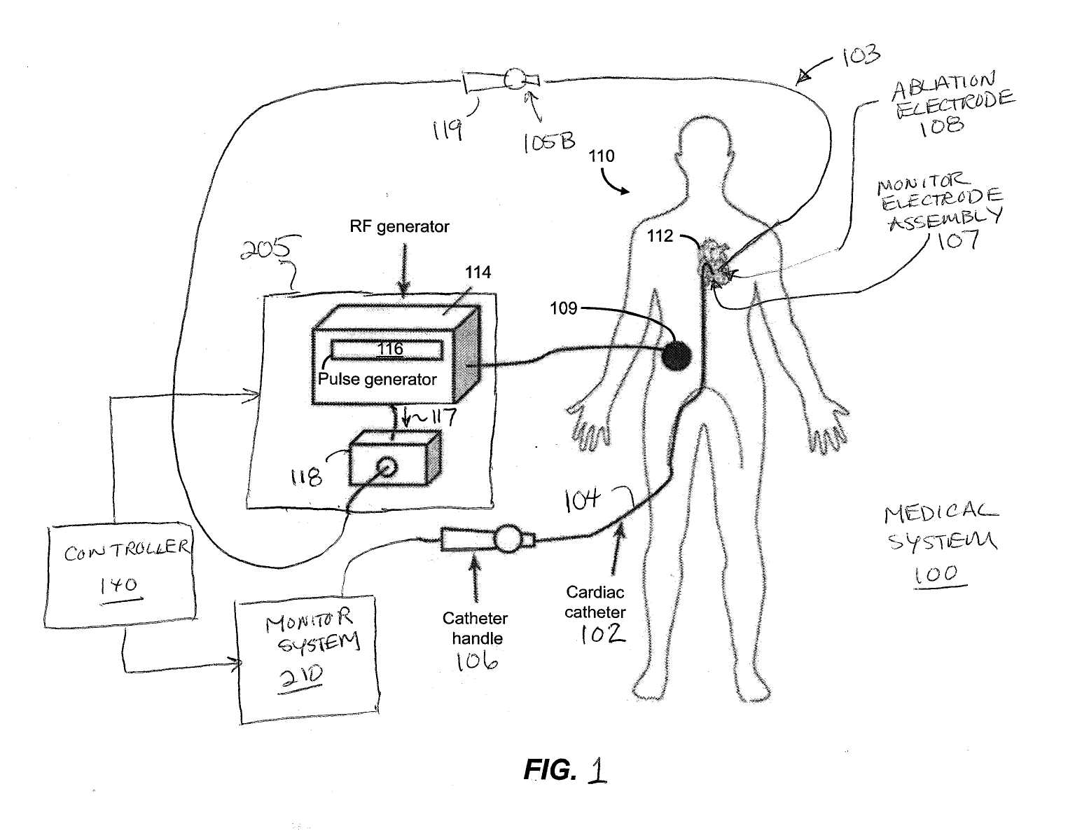

Now, more specifically, and with reference to the figures, FIG. 1 is an example diagram illustrating a medical system according to embodiments herein.

As shown, medical system 100 (such as an EP catheter system) includes a catheter 102 (first medical device), catheter 103 (second medical device), monitor system 210, an RF ablation signal generator 114, an RF amplifier 118, and at least one skin surface electrode 109.

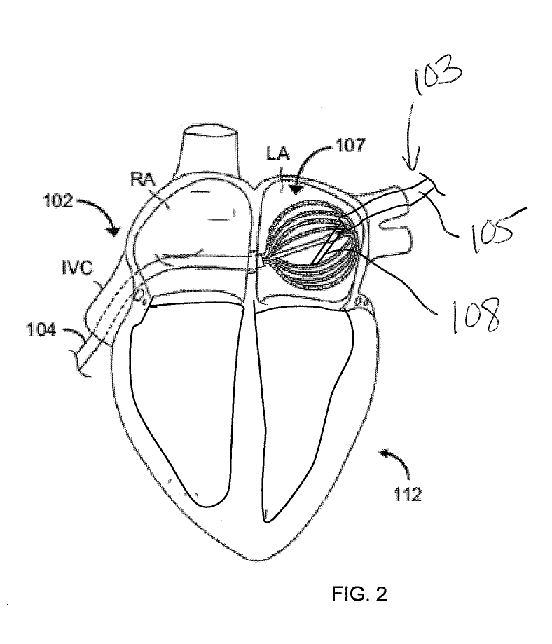

The catheter 102 can be configured as a cardiac mapping catheter 102 (see FIG. 2). The catheter 103 can be configured as a cardiac ablation catheter 103 (see FIG. 2), which is connectable to the RF ablation signal generator 114 via body 104 (cabling, flexible link between the medical site 112 and the catheter handle 106, etc.).

The RF ablation signal generator 114 includes a pulse generator 116. As its name suggests, the RF ablation signal generator 114 produces RF energy for delivery through the catheter 103 (via ablation electrode 108) to a respective medical site 112 such as a chamber in a patient's heart.

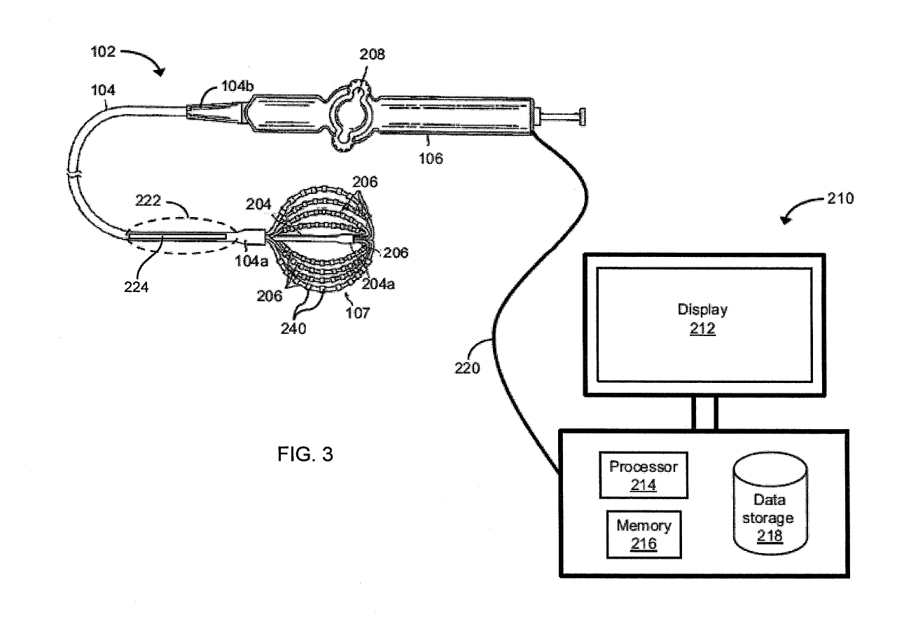

The catheter 102 configured as the cardiac mapping catheter 102 includes an elongated catheter body 104 having a proximal end 104b (see FIG. 3) and a distal end 104a of body 104 (see FIG. 3), a control handle 106 (see FIG. 3) disposed at the proximal end 104b of the catheter body 104, and an electrode assembly 107 disposed at the distal end 104a of the catheter body 104.

During operation, the monitor system 210 communicates with the electrode assembly 170. Catheter body 104 conveys signals from the monitor system 210 to the electrode assembly 107. In reverse direction, the catheter body 104 conveys signals from the electrode assembly 107 to the monitor system 210.

The catheter 103 includes an elongated catheter body 105 (cabling) having a proximal end 105b and a distal end (at the ablation electrode 108), a control handle 105 disposed at the proximal end 105b of the catheter body 105, and an ablation electrode 108 disposed at the distal end of the catheter body 105. As mentioned, the catheter 103 (operable to perform ablation) includes an elongated catheter body 105, and an ablation electrode 108 disposed at a distal end of the catheter body 105.

As shown in FIG. 2, the electrode assembly 107 can be configured as a basket-shaped electrode assembly, or as any other suitable shaped electrode assembly configuration.

In one embodiment, the electrode assembly 107 includes a plurality of intra-cardiac electrodes (such as intra-cardiac electrodes 240 to monitor attributes of tissue; see FIG. 3) disposed on a plurality of flexible spines 206 (see FIG. 3).

In an example mode of operation, the electrode assembly 107 at the distal end of the catheter body 104 is percutaneously introduced through the skin of a human subject 110 (see FIG. 1), and passed through a femoral vein or artery in the groin of the human subject 110 until the electrode assembly 107 is positioned at the medical site 112 such as in a chamber of the human subject's heart (FIG. 2).

More specifically, as shown in FIG. 2, the electrode assembly 107 may be positioned in the left atrium (LA) of the human subject's heart, and/or any other suitable location(s) within the heart. The electrode assembly 107 can present a collapsed profile or an expanded profile by manipulating, using a deflection actuator 208 (see FIG. 3), an expander segment 204 (see FIG. 3), which has a distal end 204a coupled to the plurality of flexible spines 206.

As the electrode assembly 107 is passed through the femoral vein or artery of the human subject 110, the collapsed profile of the electrode assembly 107 is guided to pass first through the inferior vena cava (IVC; see FIG. 2) to the right atrium (RA; see FIG. 2) of the heart, and then through the inter-ventricular septum of the heart to the left atrium (LA; see FIG. 2) of the heart.

After the electrode assembly 107 is positioned in the left atrium (LA) of the human subject's heart (medical site 112), the expander segment 204 is further manipulated, using the deflection actuator 208 (FIG. 3), to cause the electrode assembly 107 to transition from presenting its collapsed profile to presenting its expanded profile, as illustrated in FIG. 2.

In this example mode of operation, the electrode assembly 107 is positioned in the left atrium (LA) of the human subject's heart in order to locate one or more tissue sites of interest within the heart that are associated with arrhythmia, such as tachycardia or bradycardia arrhythmia. To that end, the cardiac mapping catheter 102 is operated to map electrical activation sequences of the heart using the plurality of intra-cardiac electrodes (such as the intra-cardiac electrodes 240; see FIG. 3) and the skin surface electrode 109, which is affixed to the skin of the human subject's body using adhesive patches.

As further shown, the catheter 102 can be communicably connected (e.g., by a cable 220; see FIG. 3) to a cardiac mapping/signal monitor 210 (see FIG. 3) in order to display one or more resulting cardiac mapping images indicating the locations of the arrhythmogenic sites within the heart. As further shown in FIG. 3, the cardiac mapping/signal monitor 210 includes at least one processor 214 (computer processor hardware, one or more computer processor devices, etc.), at least one memory 216, data storage 218, as well as a display screen 212 for displaying the cardiac mapping images.

The ablation electrode 108 at the distal end of the body 105 of the cardiac ablation catheter 103 likewise can be percutaneously introduced through the skin of the human subject 110 (see FIG. 1), and passed through another vein or artery of the human subject 110 until the electrode assembly 108 is positioned in the human subject's heart (medical sitey 112). For example, the electrode assembly 108 may be positioned in the left atrium (LA) of the human subject's heart, and/or any other suitable location(s) within the heart.

As shown in FIG. 2, the ablation electrode 108 passes through the vein or artery of the human subject 110, and is guided to the left atrium (LA; see FIG. 2) of the heart. Having located the arrhythmogenic sites mapped at monitor system 210, the ablation electrode 108 is guided within the left atrium (LA) of the human subject's heart 112 until the ablation electrode 108 is positioned proximate to each arrhythmogenic site location (specific targeted locations of tissue in the medical site 112), thereby allowing the ablation electrode 108 to ablate cardiac tissue at the arrhythmogenic site location via application of RF ablation energy from the RF ablation signal generator 114.

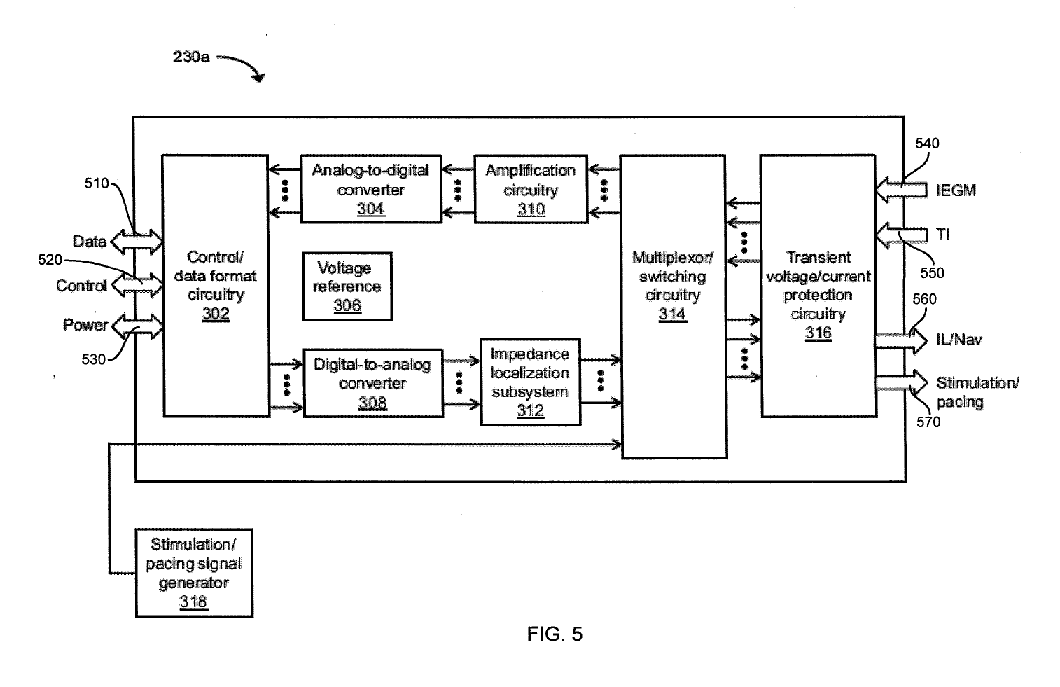

While the ablation electrode 108 ablates the cardiac tissue at the respective arrhythmogenic site locations, one or more stimulation/pacing signals generated by a stimulation/pacing signal generator 318 (see FIG. 5) is applied to the human subject's heart (such as medical site 112), as needed. Further, one or more intra-cardiac signals (e.g., IEGM signals, TI signals) is measured using one or more of the intra-cardiac electrodes 240 (such as metal oxide electrodes) of the electrode assembly 107. The cable 220 conveys the intra-cardiac signal measurements to monitor system 210 where the signals are monitored, used to produce a map, etc.

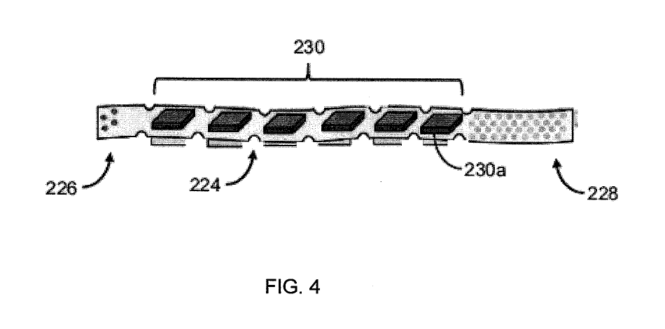

In one example embodiment, to minimize the need for cabling through the catheter body 104 for accessing and monitoring the intra-cardiac signal measurements generated from the plurality of intra-cardiac electrodes (e.g., the intra-cardiac electrodes 240), the cardiac mapping catheter 102 can be configured to include one or more integrated circuits 230 (see FIG. 4) on respective flexible strips disposed in a region 222 (see FIG. 3) of the catheter body 104 (flexible link) toward its distal end 104a in proximity to the monitor electrode assembly 107. Note that region 222 is shown by way of example only; the flexible strip 224 can be disposed at any suitable location.

As further shown in FIG. 4, the plurality of integrated circuits 230 are electrically and mechanically connected to a flex strip 224, which includes a first set of conductive pads 226 for interfacing the respective integrated circuits 230 over cable 220 with the cardiac mapping/signal monitor 210. A second set of conductive pads 228 of the flex strip 224 provides electrical connections between the respective integrated circuits 230 and the multitude of intra-cardiac electrodes (e.g., the intra-cardiac electrodes 240) included in the electrode assembly 107.

Accordingly, in one embodiment, the catheter 102 (such as a medical device) includes an electrode assembly 107 and body 104 (i.e., a flexible link). As previously discussed, the electrode assembly 107 is operable to monitor tissue at the medical site 112. The integrated circuitry (one or more integrated circuits 230 on flex strip 224) is disposed in the body 104 (flexible link) and is electrically coupled to the electrode assembly to convey signals received from the monitor system 210 through body 104 to the medical site 112. Additionally, the integrated circuitry (one or more integrated circuits 230) is operable to convey signals from the medical site 112 through body 104 to the monitor system 210.

FIG. 5 depicts an example integrated circuit 230a from among the plurality of integrated circuits 230 of FIG. 4. It is noted that each of the plurality of integrated circuits 230 in FIG. 4 is configured and arranged in a similar manner and provides similar functionality as the integrated circuit 230a shown in FIG. 5.

As shown in FIG. 5, the integrated circuit 230a includes a plurality of functional modules, including control/data format circuitry 302, an Analog-to-Digital Converter (ADC) 304, a voltage reference 306, a digital-to-analog converter (DAC) 308, amplification circuitry 310, an impedance localization (IL) subsystem 312, multiplexor/switching circuitry 314, and transient voltage/current protection circuitry 316.

The transient voltage/current protection circuitry 316 is operable to protect the functional modules within the integrated circuit 230a from electrostatic discharge (ESD) caused by high RF ablation and/or defibrillation energy waves.

It is further noted that the integrated circuit 230a of FIG. 5 is described herein for purposes of illustration, and that alternative embodiments of the integrated circuit 230a includes one or more additional functional modules, one or more fewer functional modules, and/or at least one modification to one or more of the functional modules 302, 304, 306, 308, 310, 312, 314, 316.

For example, in certain alternative embodiments, one or more amplification stages may be provided between the multiplexor/switching circuitry 314 and the transient voltage/current protection circuitry 316. In certain further alternative embodiments, filtering functionality may be included in the transient voltage/current protection circuitry 316.

As shown, integrated circuit 230a includes input for power channel 530. Monitor system 210 conveys power signals over power channel 530 of cable 220 and body 104 to power each of the integrated circuits 230a on strip 224.

During the mapping of the electrical activation sequences of the human subject's heart (such as medical site 112), the cardiac mapping/signal monitor 210 provides cardiac mapping/navigation signals (also referred to herein as the “IL/Nav signals”) over a data channel 510 to the control/data format circuitry 302, which provides the IL/Nav signals to the DAC 308 (Digital to Analog Converter).

The DAC 308 converts the IL/Nav signals from digital form to analog form, and provides the IL/Nav signals in analog form to the IL (Impedance Localization) subsystem 312. The IL subsystem 312 uses the IL/Nav signals to determine positions of the electrode assembly 107 within the human subject's heart using any suitable impedance-based position-sensing techniques.

Once processed by the impedance localization subsystem 312, the IL/Nav signals are provided to the multiplexor/switching circuitry 314, which, in turn, provides the IL/Nav signals, via the transient voltage/current protection circuitry 316, over an IL/Nav channel 560 to some or all of the intra-cardiac electrodes (e.g., the intra-cardiac electrodes 240) of electrode assembly 107.

As further shown, the stimulation/pacing signal generator 318 likewise provides stimulation/pacing signals to the multiplexor/switching circuitry 314, which, in turn, provides the stimulation/pacing signals, via the transient voltage/current protection circuitry 316, over a simulation/pacing channel 570 to one or more of the intra-cardiac electrodes (e.g., the intra-cardiac electrodes 240) of the electrode assembly 107.

It is noted that the cardiac mapping/signal monitor 210 controls the switching of the multiplexor/switching circuitry 314 by providing suitable control signals over control channel 520 to the control/data format circuitry 302.

During the ablation of the cardiac tissue at the arrhythmogenic site locations, the cardiac mapping/signal monitor 210 monitors one or more intra-cardiac signal measurements (e.g., IEGM signal measurements, TI signal measurements) provided from one or more of the intra-cardiac electrodes (e.g., the intra-cardiac electrodes 240) of the electrode assembly 107. To that end, such IEGM signal measurements and TI signal measurements is provided over an IEGM channel 540 and a TI channel 550, respectively, to the transient voltage/current protection circuitry 316, which, in turn, provides the IEGM and TI signal measurements to the multiplexor/switching circuitry 314.

Under the control of the cardiac mapping/signal monitor 210, the multiplexor/switching circuitry 314 provides the IEGM and/or TI signal measurements to the amplification circuitry 310, which amplifies the low amplitude IEGM and/or TI signals, and provides the amplified IEGM and/or TI signals to the ADC 304.

The ADC 304 converts the amplified IEGM and/or TI signals from analog form to digital form, and provides the IEGM and/or TI signal measurements in digital form to the control/data format circuitry 302, which, in turn, provides the IEGM and/or TI signal measurements over the Data channel 510 to the cardiac mapping/signal monitor 210 for subsequent processing and display of the IEGM and/or TI signal measurement waveforms on the display screen 212.

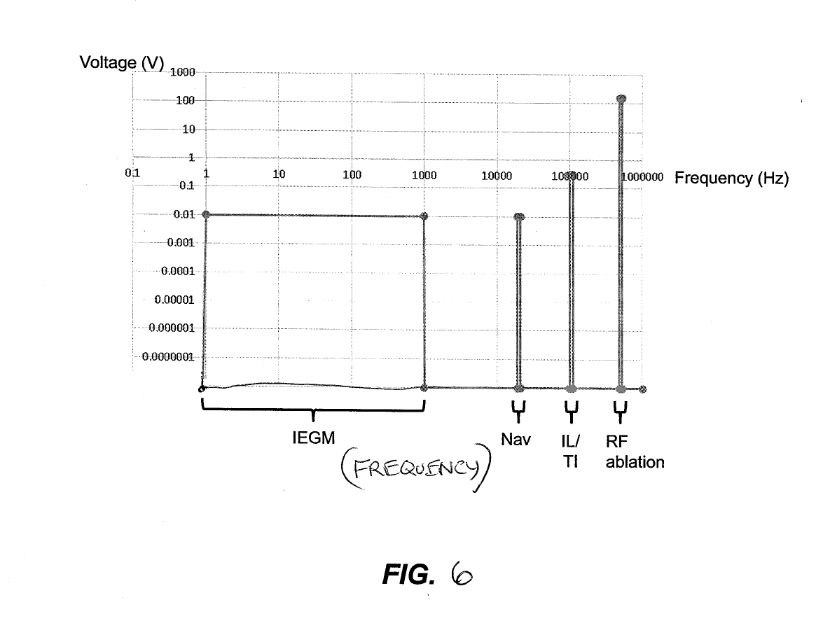

FIG. 6 depicts, according to embodiments herein, example amplitudes and frequencies corresponding to the IEGM signals, the Nav signals, the IL signals, the TI signals, and the RF ablation signals according to embodiments herein. Note that frequency and amplitude characteristics may vary depending upon different embodiments.

As shown in FIG. 6, such IEGM signals have amplitudes of up to 10 mV (millivolts) within a frequency range of about 1 to 1,000 Hz, such Nav signals can have amplitudes of up to 10 mV at a frequency of about 20,000 Hz, such IL/TI signals can have amplitudes of about 300 mV within a frequency range of about 100,000 to 110,000 Hz, and such RF ablation signals can have amplitudes of about 150 to 300 Vpeak-peak within a frequency range of about 480,000 to 520,000 Hz.

As mentioned, these frequency and amplitude characteristics of respective signals may vary depending upon the embodiment.

It is noted that unwanted interference from the high amplitude RF ablation signals are optionally coupled onto the lower amplitude intra-cardiac signals (e.g., the IEGM and/or TI signals) during the ablation of cardiac tissue, often necessitating the use of large and costly filter components to remove such interference signals from the intra-cardiac signals.

In order to obviate the need for such large and costly filter components, the ablation electrode 108 of the cardiac ablation catheter 103 applies RF ablation energy to the cardiac tissue at arrhythmogenic site locations in ablation time windows, each of which delivers a series of high amplitude RF ablation pulses to the medical site 112.

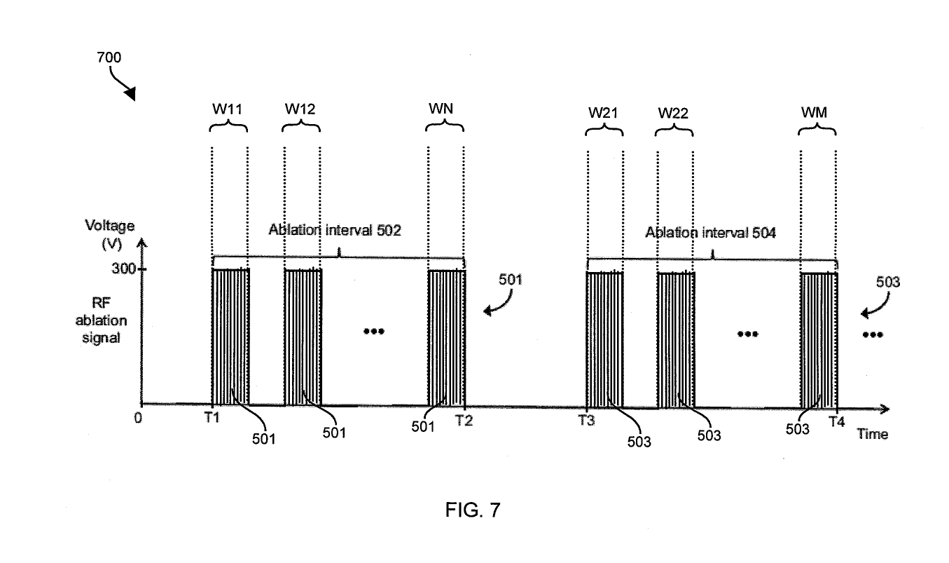

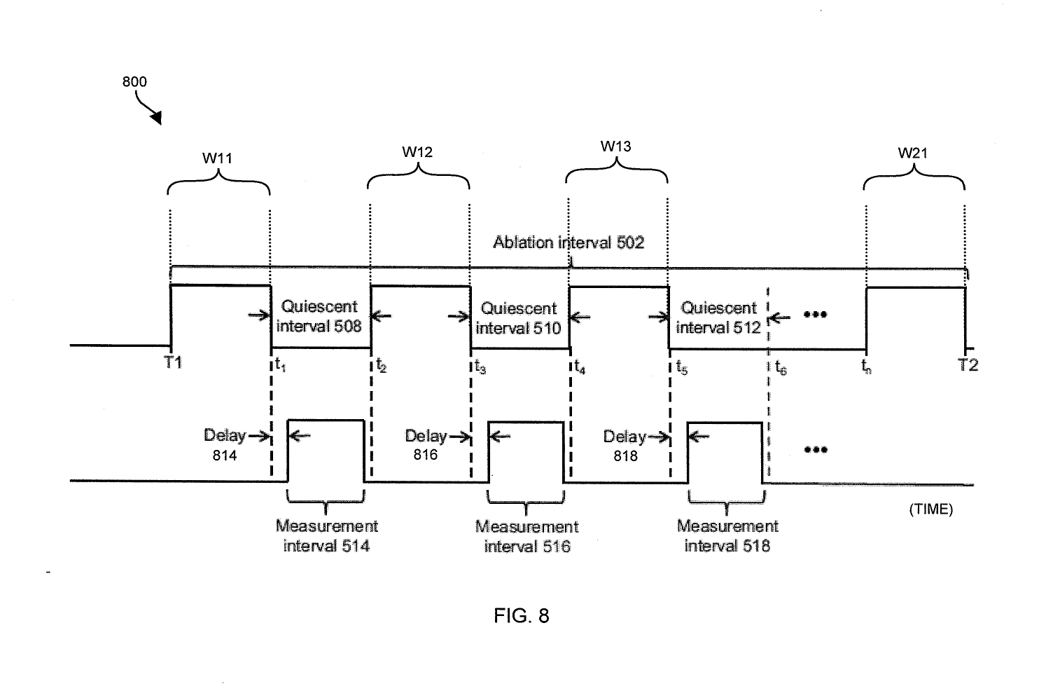

Further, the plurality of intra-cardiac electrodes (e.g., the intra-cardiac electrodes 240) of the cardiac mapping catheter 102 makes and/or monitors intra-cardiac signal measurements (e.g., the IEGM and/or TI signal measurements) during one or more quiescent intervals (non-ablation windows) between the respective RF ablation pulses. Note that details of the ablation intervals and quiescent intervals or further discussed with respect to FIGS. 7 and 8.

Referring again to FIG. 1, in one embodiment, the pulse generator 116 within the RF ablation signal generator 114 is optionally configured to generate electrical pulses that the RF ablation signal generator 114 uses to pulse its RF ablation energy output 117 “ON” and “OFF.”

The RF ablation signal generator 114 provides the pulsed RF ablation energy 117 to the RF amplifier 118, which amplifies the pulsed RF ablation energy 117 to produce each series of high amplitude RF ablation pulses.

In accordance with control input from the controller 140, the system 205 (including the RF generator 114 and amplifier 118) conveys the series of RF ablation pulses through the catheter body 105 (such as a flexible cable) to the ablation electrode 108 of the cardiac ablation catheter 103 for use in ablating tissue at a respective medical site 112 (such as a heart).

As further described herein, while the targeted tissue at the medical site is being ablated, or in between operations of performing ablation, the cardiac mapping/signal monitor 210 provides suitable control signals to one or more of the plurality of integrated circuits 230 to control the switching of the multiplexor/switching circuitry 314, thereby assuring that the intra-cardiac signal measurements (e.g., the IEGM and/or TI signal measurements), if needed, are being made and/or monitored during the quiescent intervals between the respective RF ablation intervals.

FIG. 7 depicts an exemplary RF ablation signal that includes a series of high amplitude RF ablation pulses according to embodiments herein.

According to one embodiment, the controller 140 as described herein coordinates use of multiple medical devices at a medical site 112. In one embodiment, to coordinate use of the catheter 102 and catheter 103, the controller 140 implements the control sequence (windows in timing diagram 700) to control switching between different operational modes such as a first mode of enabling the catheter 103 to deliver ablation pulses to the medical site 112 and a second mode of disabling the catheter 103 from delivering ablation pulses to the medical site 112.

As shown in FIG. 7, assume that a respective caregiver operating the catheter 103 provides input to perform an ablation process on particular selected tissue at the medical site 112. The caregiver steers the ablation electrode 108 to the appropriate site to be ablated. In one embodiment, the caregiver operating the catheter 102 additionally provides input to perform a non-ablation operations such as monitoring the particular tissue being ablated (or other tissue) at the medical site 112.

In this example embodiment, in accordance with the input from the caregiver to perform the ablation operation on the particular selected tissue (such as by contacting the ablation electrode 108 to the selected tissue and pressing a button on the handle 119 or other suitable trigger), the ablation electrode 108 delivers a series of high amplitude RF ablation pulses 501 during windows W11, W12, . . . , Wn between time T1 and T2.

Thus, in accordance with input from a caregiver to perform a single ablation operation such as by activating a trigger, the controller 140 enables or causes the ablation electrode 108 to deliver a respective sequence of energy pulses during each window of time W11, W12, W13, etc., such that the catheter device 103 delivers discontinuous delivery of multiple windows of energy pulses to target tissue at the medical site 112 to perform a single ablation operation during ablation interval 502.

In one embodiment, each of the high amplitude RF ablation pulses asserted in the respective ablation intervals 502, 504 optionally has an amplitude level of about 150 to 300 Vp-p, or any other suitable amplitude level. In one embodiment, a respective caregiver determines how much energy over time should be delivered to tissue to perform a respective ablation operation. Taking into account the duty cycle of activating the respective windows W11, W12, etc., the peak to peak voltage of energy pulses can be adjusted up or down such that the overall energy delivered between time T1 and T2 is appropriate for performing a desired ablation operation.

Further, note that the frequency of the plurality of ablation intervals (such as ablation intervals 502, 504) may be within a frequency range of about 480,000 to 520,000 Hertz, or any other suitable frequency range.

In accordance with yet further embodiments, each respective set of ablation pulses in a respective ablation window can be configured to include an integer number of energy pulses (integer number of complete periods or cycles) to deliver to the tissue at the medical site 112 to reduce generation of RF (Radio Frequency) noise at the medical site. In other words, embodiments herein can include precisely controlling attributes (phase angle start and stop times, number of pulses, amplitude, etc.) of the energy pulses used to perform the ablation operation in order to reduce the amount of noise at the medical site 112 so that the monitor electrode assembly 107 does not experience RF interference.

Note that the frequency of the RF ablation pulses 501 (such as 250-750 KHz) is substantially greater than the frequency of a respective ablation interval 502 (such as 1-3 KHz). Accordingly, the ablation electrode 108 is operable to deliver a sequence of multiple ablation pulses in each of windows W11, W12, Wn, etc., to perform an ablation operation on first selected tissue at the medical site 112. The ablation electrode 108 is operable to deliver a sequence of multiple ablation pulses in each of windows W21, W22, Wm, etc., to perform an ablation operation on second selected tissue at the medical site 112.

As further shown below, non-ablation operations can be performed during quiescent intervals between the ablation intervals (such as window W11, W12, etc.).

FIG. 8 is an example diagram illustrating switching between ablation intervals and quiescent intervals to perform ablation and non-ablation operations according to embodiments herein.

Timing diagram 800 depicts an example timing (as provided by controller 140) of the series of high amplitude RF ablation pulses 501 asserted in the ablation interval 502 relative to timing of measurement intervals 514, 516, 518, etc., during which intra-cardiac signal measurements are made and/or monitored. As shown, the quiescent intervals 508, 510, 512, etc., occur between the ablation windows W11, W12, W13, etc.

More specifically, the controller 140 controls operation of the catheter 102 and the catheter 103 such that the quiescent interval 508 for performing non-ablation operations occurs between window W11 and window W12, the quiescent interval 510 occurs between window W12 and window W13, and so on.

Accordingly, via respective control windows, the controller 140 spaces apart the windows W11, W12, W13, etc., of operating in the ablation mode via quiescent intervals 508, 510, etc., such that the catheter 103 is disabled from applying continuous pulses of energy to the tissue at the medical site 112 in favor of providing the quiescent intervals 508, 510, 512, etc, to perform non-ablation operations such as measurements of the tissue being ablated (or tissue not being ablated) during the windows W11, W12, W13, etc.

In accordance with a caregiver operating the catheter 103 to deliver ablation energy to the medical site 112 using ablation electrode 108, the series of high amplitude RF ablation pulses 501 includes a first sequence (window W11) of electrical pulses delivered to the medical site 112 between time T1 to time t1, a second sequence (window W12) of electrical pulses delivered to the medical site 112 between time t2 to time t3, and so on, to a last electrical pulse asserted from time to to time T2.

As previously discussed, the quiescent interval 508 occurs from time t1 to time t2, quiescent interval 510 occurs from time t3 to time t4, quiescent interval 512 occurs from time t5 to time t6, and so on, to a last quiescent interval (not shown) that occurs from time tn−1 to time tn.

Thus, as described herein, while selected tissue at the medical site 112 is being ablated during ablation intervals, the cardiac mapping/signal monitor 210 provides suitable control signals to control the switching of the multiplexor/switching circuitry 314, thereby assuring that intra-cardiac signal measurements (e.g., IEGM and/or TI signal measurements) are being made and/or monitored during quiescent intervals in between respective windows of RF ablation pulses. In one embodiment, the quiescent intervals during which the intra-cardiac signal measurements (e.g., the IEGM and/or TI signal measurements) are being made and/or monitored include the quiescent intervals 508, 510, 512 shown in timing diagram 800.

In accordance with further embodiments, the cardiac mapping/signal monitor system 210 controls the switching of the multiplexor/switching circuitry 314 to assure that such intra-cardiac signal measurements are being made and/or monitored during the measurement intervals 514, 516, 518 that occur within the quiescent intervals 508, 510, 512, respectively. For example, in this embodiment, the intra-cardiac signal measurements can be made and/or monitored during the respective measurement intervals 514, 516, 518, and so on, at a sampling rate that is greater than the appropriate Nyquist frequency to collect respective data.

Note that, as needed, the duty cycle of ablation windows and non-ablation windows (quiescent intervals 508, 510, 512, etc.) can be adjusted such that sufficient time is allotted to the monitoring system including monitor electrode assembly 107 to take measurements and collect appropriate data for notification to the respective caregiver.

In accordance with further embodiments, the cardiac mapping/signal monitor system 210 further controls the switching of the multiplexor/switching circuitry 314 to delay (via delay 814 in the quiescent interval 508, delay 816 in the quiescent interval 510, delay 818 in the quiescent interval 512, etc.) the generation of and/or monitoring of the intra-cardiac signal measurements. The delays help to ensure that dissipation of the RF energy pulses so that they do not interfere with the measurements.

In another embodiment, as shown in FIG. 7, the quiescent intervals during which the intra-cardiac signal measurements (e.g., the IEGM and/or TI signal measurements) are being made and/or monitored further includes a quiescent interval spanning from time T2 to time T3 between the respective ablation intervals 502, 504.

By providing the functional modules 302, 304, 306, 308, 310, 312, 314, 316 within the respective integrated circuits 230 in the region 222 of the body 104 of the cardiac mapping catheter 102, and making and/or monitoring intra-cardiac signal measurements during measurement intervals between adjacent RF ablation pulses, the size of the electronics required per electrode of the electronic assembly 107 can be reduced, thereby allowing for higher channel counts in the cardiac mapping catheter 102 for monitoring the intra-cardiac signal measurements and ultimately resulting in better cardiac images.

Accordingly, to summarize one embodiment, during windows of operating in an ablation mode (such as window W11, W12, etc., as previously discussed), the controller 140 enables the monitor ablation electrode 108 to perform an ablation operation; during windows of time operating in a non-ablation mode (such as during quiescent intervals 508, 510, 512, etc.), the controller 140 disables the catheter 103 from performing the ablation operation. Accordingly, because the catheter 103 is disabled (prevented by controller 140) from performing the ablation operation during quiescent intervals 508, 510, 512, etc., the catheter 102 and corresponding monitor electrode assembly 107 are able to perform functions without interference from the ablation operation being performed by the catheter 103. As previously discussed, in one embodiment, the monitor electrode assembly 107 is optionally a multi-sensor device including multiple sensors. Each of the sensors monitors respective tissue at a different location of the medical site 112 in which the ablation electrode 108 performs ablation. In one embodiment, the multi-sensor device is activated to collect data during windows of time such as measurement intervals 514, 516, 518, etc.

Note further that a magnitude of the energy pulses delivered by the ablation electrode 108 via catheter 103 is sufficiently high such that the discontinuous delivery of multiple windows of energy pulses (W11, W12, W13, etc.,) provides the same ablation results as if the catheter 103 were operated in a continuous mode to ablate the tissue at medical site 112. In other words, each of the windows of operating in the (non-ablation mode such as quiescent intervals 508, 510, 512, etc.) are sufficiently short in time that the corresponding tissue being ablated does not have time to substantially cool, resulting in ablation similar to ablation that would otherwise occur in a continuous operational mode with no quiescent intervals.

In such an instance, because the amount of energy supplied by the ablation electrode 108 of catheter 103 is elevated to account for operating in the discontinuous mode, the catheter 103 supplies a sufficiently high amount to account for the corresponding OFF time (quiescent intervals). In other words, the energy of the pulses delivered in ablation windows is elevated such that an appropriate amount of energy per unit time is provided to perform the ablation operation.

In accordance with further embodiments, note that the quiescent intervals 508, 510, 512, etc., are sufficiently short in time that the thermal decay (drop in temperature) of tissue being ablated at the medical site 112 when no ablation energy is delivered during the quiescent intervals is insignificant.

In accordance with further embodiments, each ablation window W11, W12, etc., is typically selected as a value between 5 microseconds and 100 milliseconds, or any other suitable value; each quiescent interval is typically selected as a value between 5 microseconds and 100 milliseconds, or any other suitable value. Energy pulses in the windows can be any suitable duration such as 0.1 to 10 microseconds, or any other suitable value.

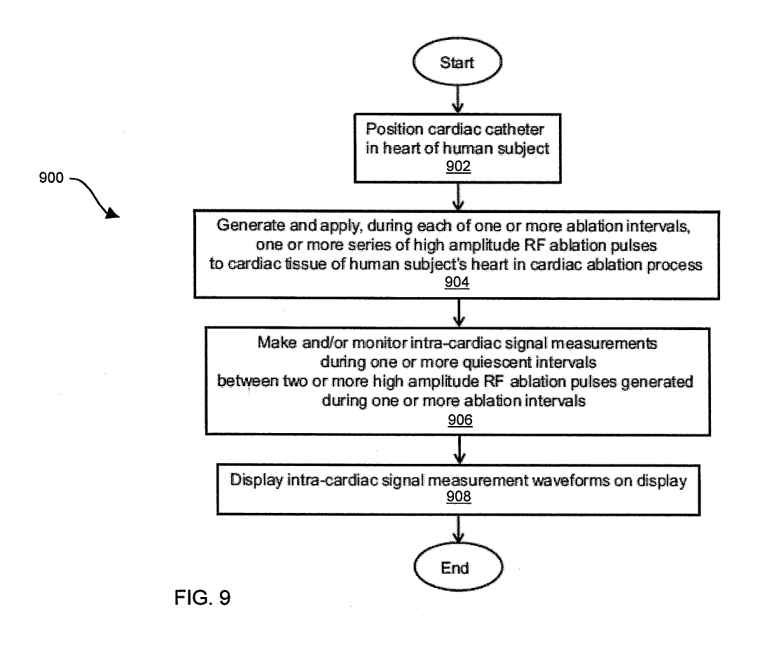

A further method of performing cardiac ablation is described below in flowchart 900 (FIG. 9) with reference to FIGS. 1, 2, 8, and 9.

As depicted in block 902 of flowchart 900, the catheter 103 (a first medical device) and catheter 102 (a second medical device) are simultaneously positioned to be located at the medical site such as a chamber of the medical site 112 of the human subject 110.

As depicted in block 904, the amplifier 118 produces a sequence of RF ablation pulses during each of one or more ablation intervals 502, 504, etc. The catheter body 105 conveys the high amplitude RF ablation pulses during each window in the ablation intervals to the ablation electrode 108 to perform an ablation operation on tissue (such as tissue in the heart).

As depicted in block 906, intra-cardiac signal measurements are made and/or monitored, by the cardiac mapping/signal monitor 210 (see FIG. 3), during one or more quiescent intervals 508, 510, 512, etc. As previously discussed, each quiescent interval occurs between two corresponding ablation windows in which RF ablation pulses are delivered to the target tissue via the ablation electrode 108.

As depicted in block 908, the monitor system 210 displays intra-cardiac signal measurement waveforms on a respective display screen 212, allowing the caregiver to view collected data/measurements obtained during the quiescent intervals.

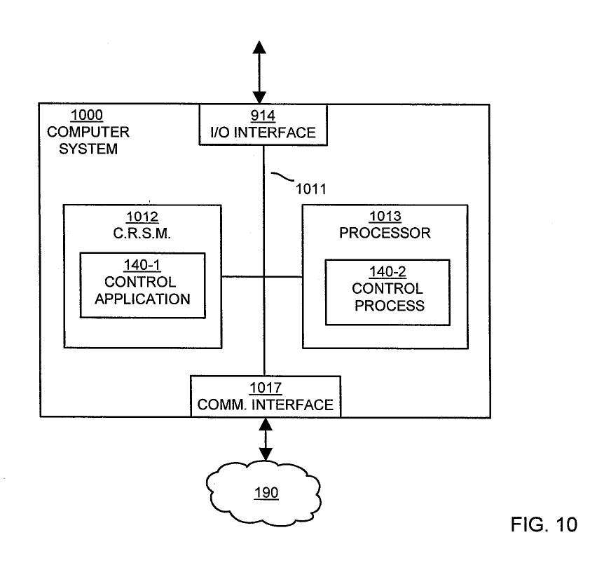

FIG. 10 is an example block diagram of a computer apparatus for implementing any of the operations as discussed herein according to embodiments herein.

Any of the resources (e.g., the controller 140, system 205, monitor system 210, catheter 102, catheter 103, etc.) can be configured to include a processor and executable instructions to carry out the different operations as discussed herein.

As shown, computer system 1000 of the present example includes an interconnect 1011 that couples computer readable storage media 1012 such as a non-transitory type of media (i.e., any type of hardware storage medium) in which digital information can be stored and retrieved, a processor 1013 (computer processor hardware), I/O interface 1014, etc.

Computer readable storage medium 1012 can be or include any hardware storage device such as memory, optical storage, hard drive, floppy disk, etc. In one embodiment, the computer readable storage medium 1012 stores instructions and/or data.

As shown, computer readable storage media 1012 can be encoded with control application 140-1 (e.g., including instructions) to carry out any of the operations as discussed herein.

During operation of one embodiment, processor 1013 (computer processor hardware) accesses computer readable storage media 1012 via the use of interconnect 1011 in order to launch, run, execute, interpret or otherwise perform the instructions in control application 140-1 stored on computer readable storage medium 1012. Execution of the control application 140-1 produces control process 140-2 to carry out any of the operations and/or processes as discussed herein.

Those skilled in the art will understand that the computer system 1050 can include other processes and/or software and hardware components, such as an operating apparatus that controls allocation and use of hardware resources to control application 140-1.

In accordance with different embodiments, note that computer apparatus may be or included in any of various types of devices, including, but not limited to, a mobile computer, a personal computer apparatus, a wireless device, base station, phone device, desktop computer, laptop, notebook, net book computer, mainframe computer apparatus, handheld computer, workstation, network computer, application server, storage device, a consumer electronics device such as a camera, camcorder, set top box, mobile device, video game console, handheld video game device, a peripheral device such as a switch, modem, router, set-top box, content management device, handheld remote control device, any type of computing or electronic device, etc.

The computer system 1000 may reside at any location or can be included in any suitable one or more resources in a network environment to implement functionality as discussed herein.

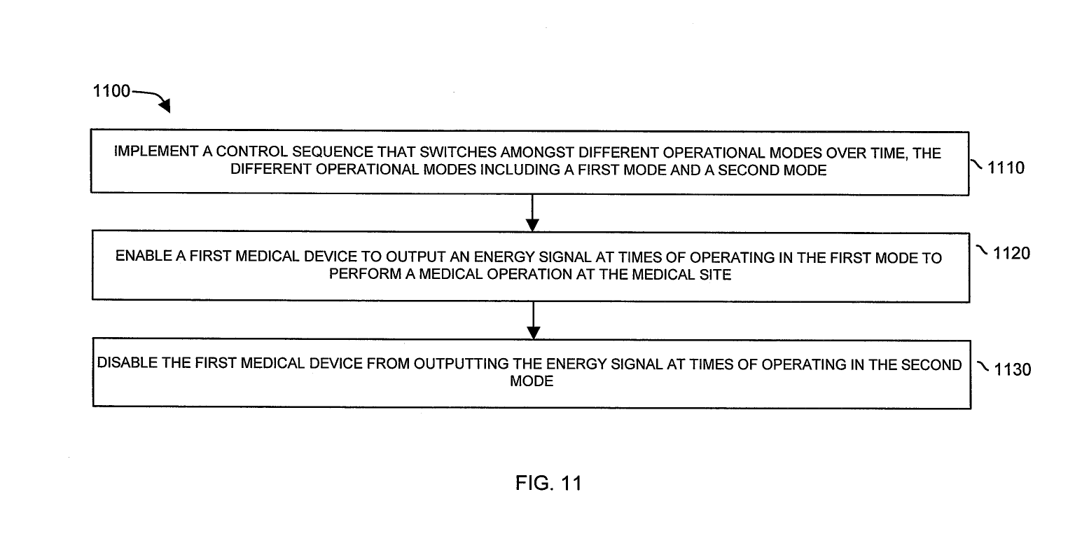

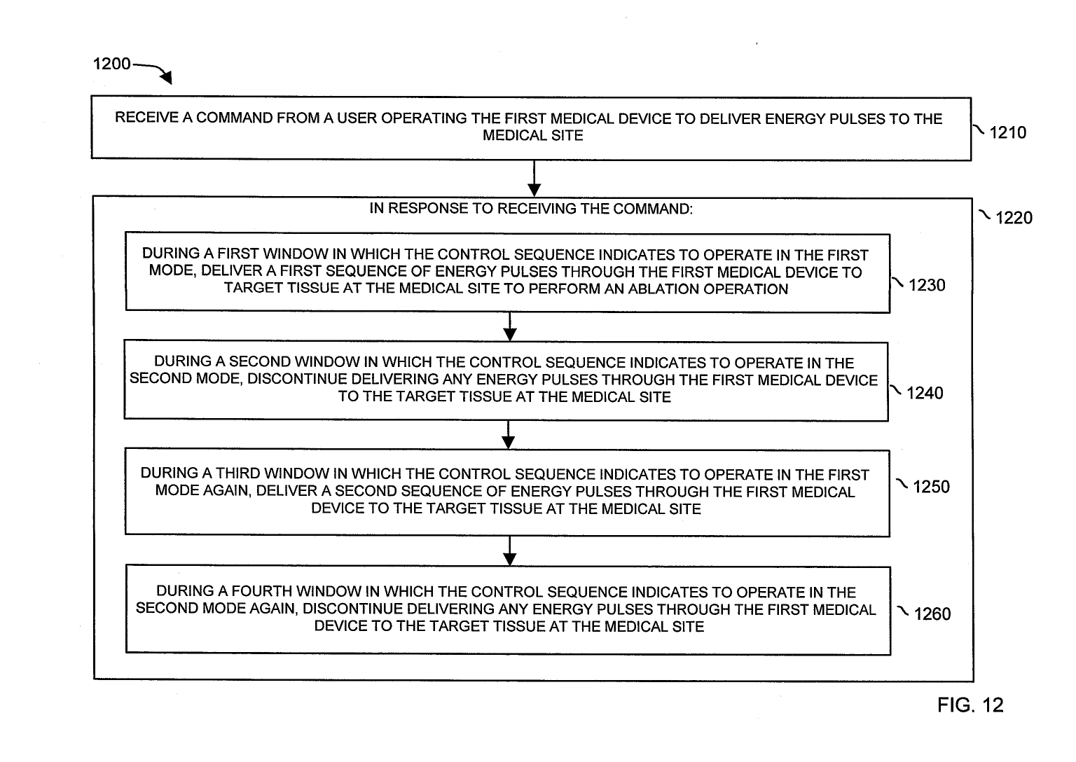

Functionality supported by the different resources will now be discussed via flowcharts in FIGS. 11-12. Note that the steps in the flowcharts below can be executed in any suitable order.

FIG. 11 is a flowchart 1100 illustrating an example method according to embodiments. Note that there will be some overlap with respect to concepts as discussed above.

In processing operation 1110, the controller 140 implements a control sequence that switches amongst different operational modes over time. The different operational modes include a first mode (mode to perform ablation of tissue) and a second mode (mode to perform non-ablation operations). In one embodiment, as previously discussed, the sequence includes first windows of time (such as windows W11, W12, etc.,) of operating in the first mode (such as to perform ablation) and second windows of time (such as quiescent intervals 508, 510, 512, etc.) of operating in the second mode (such as to perform non-ablation operations).

In processing operation 1120, the controller 140 enables a first medical device to output an energy signal during windows of time (such as windows W11, W12, etc.) of operating in the first mode to perform a medical operation at the medical site 112. In processing operation 1130, the controller 140 disables the first medical device from outputting the energy signal during windows of time of operating in the second mode.

FIG. 12 is a flowchart 1200 illustrating an example method according to embodiments. Note that there will be some overlap with respect to concepts as discussed above.

In processing operation 1210, the controller 140 receives a command from a user operating the first medical device (such as catheter 103) to deliver energy pulses to the medical site 112.

In processing operation 1220, the controller 140 switches between operating in a first mode (windows W11, W12, etc.) and a second mode (quiescent intervals 514, 516, etc.).

In processing operation 1230, during a first window (W11) in which the control sequence (as in FIGS. 7 and 8) indicates to operate in the first mode, the system 205 delivers a first sequence of energy pulses through the catheter 103 to target tissue at the medical site 112 to perform an ablation operation.

In processing operation 1240, during a second window (such as quiescent interval 508) in which the control sequence indicates to operate in the second mode (such as a measurement mode), the catheter 103 discontinues delivering any energy pulses to the target tissue at the medical site 112.

In processing operation 1250, during a third window (W12) in which the control sequence indicates to operate in the first mode again, the catheter 103 delivers a second sequence of energy pulses through to the target tissue at the medical site 112.

In processing operation 1260, during a fourth window (such as quiescent interval 510) in which the control sequence indicates to operate in the second mode again, the catheter 103 discontinues delivering any energy pulses to the target tissue at the medical site 112.

Note again that techniques herein are well suited to support coordination/control of multiple medical devices at a medical site. However, it should be noted that embodiments herein are not limited to use in such applications and that the techniques discussed herein are well suited for other applications as well.

Based on the description set forth herein, numerous specific details have been set forth to provide a thorough understanding of claimed subject matter. However, it will be understood by those skilled in the art that claimed subject matter may be practiced without these specific details. In other instances, methods, apparatuses, apparatuses, etc., that would be known by one of ordinary skill have not been described in detail so as not to obscure claimed subject matter. Some portions of the detailed description have been presented in terms of algorithms or symbolic representations of operations on data bits or binary digital signals stored within a computing apparatus memory, such as a computer memory. These algorithmic descriptions or representations are examples of techniques used by those of ordinary skill in the data processing arts to convey the substance of their work to others skilled in the art. An algorithm as described herein, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has been convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing platform, such as a computer or a similar electronic computing device, that manipulates or transforms data represented as physical electronic or magnetic quantities within memories, registers, or other information storage devices, transmission devices, or display devices of the computing platform.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

We claim:

1. A system to coordinate use of multiple medical devices at a medical site, the system comprising:
a first medical device to perform an ablation operation at the medical site;
a second medical device to perform non-ablation operations at the medical site; and
a controller operable to control switching between different operational modes including a first mode and a second mode, the first mode enabling the first medical device to perform the ablation operation, the second mode operable to disable the first medical device from performing the ablation operation.

2. The system as in claim 1, wherein the first medical device is operable to deliver, during spaced apart windows of operating in the first mode, energy pulses to the medical site to perform the ablation operation.

3. The system as in claim 1, wherein the second medical device is prevented from performing non-ablation operations during the first mode.

4. The system as in claim 1, wherein the second medical device is a multi-sensor device including multiple sensors, each of which is operable to monitor respective tissue at a different location of the medical site.

5. The system as in claim 4 further comprising:
a display screen operable to display detected attributes of tissue at the medical site as sensed by the multiple sensors, the attributes of the tissue detected by the multi-sensor device during spaced apart windows of time of operating in the second mode.

6. The system as in claim 1 further comprising:
a signal generator resource to produce ablation pulses to ablate tissue at the medical site, the first medical device operable to deliver a respective set of multiple ablation pulses generated by the generator resource during each of multiple windows of time of operating in the first mode.

7. The system as in claim 6, wherein each respective set of ablation pulses includes a corresponding integer number of energy pulses to deliver to the tissue at the medical site.

8. The system as in claim 1, wherein the controller is further operable to:
in response to receiving a command from a user operating the first medical device to deliver energy pulses to the medical site:
during a first window time of operating in the first mode, deliver a first sequence of energy pulses through the first medical device to tissue at the medical site to perform the ablation operation;
during a second window of time of operating in the second mode, discontinue delivering any energy pulses through the first medical device to the tissue at the medical site, the second mode pausing the ablation operation by the first medical device; and
during a third window of time of operating in the first mode, deliver a second sequence of energy pulses through the first medical device to the tissue at the medical site to continue the ablation operation.

9. The system as in claim 1, wherein the controller is further operable to:
delay activation of the second medical device with respect to a time of transitioning from operating in the first mode to operating in the second mode.

10. The system as in claim 1 further comprising;
a filter resource to filter feedback received from the second medical device based on windows of time in which the control sequence indicates to operate in the first mode.

11. A method to control use of multiple medical devices at a medical site, the method comprising:
implementing a control sequence that switches between different operational modes over time, the different operational modes including a first mode and a second mode;
enabling a first medical device to output an energy signal during windows of time of operating in the first mode to perform an ablation operation on tissue at the medical site; and
disabling the first medical device from outputting the energy signal during windows of time of operating in the second mode.

12. The method as in claim 11 further comprising:
during the first mode, enabling delivery of a respective sequence of energy pulses during each window of time of operating in the first mode such that the first medical device delivers spaced apart delivery of multiple windows of energy pulses to the medical site to perform the ablation operation.

13. The method as in claim 11 further comprising:
disabling the second medical device from performing non-ablation operations during the first mode.

14. The method as in claim 11 further comprising:
enabling the second medical device during windows of time of operating in the second mode, the second medical device being a multi-sensor device including multiple sensors, each of which is operable to monitor respective tissue at the medical site.

15. The method as in claim 14 further comprising:
displaying detected attributes of different locations of the medical site as sensed by the multiple sensors, the attributes of the different locations detected by the multi-sensor device while in the second mode, which occurs in between windows of time of operating in the first mode.

16. The method as in claim 11 further comprising:
producing ablation pulses to ablate tissue at the medical site, the first medical device operable to deliver a respective set of multiple ablation pulses during each of multiple windows of time of operating in the first mode.

17. The method as in claim 16, wherein the respective set of ablation pulses includes an integer number of energy pulses to deliver to the tissue at the medical site.

18. The method as in claim 11 further comprising:
in response to receiving a command from a user operating the first medical device to deliver energy pulses to the medical site:
during a first window of operating in the first mode, deliver a first sequence of energy pulses via the first medical device to the tissue at the medical site to perform the ablation operation;
during a second window operating in the second mode, discontinue delivering any energy pulses via the first medical device to the tissue at the medical site, the second mode interrupting the ablation operation by the first medical device; and
during a third window of time of operating in the first mode, deliver a second sequence of energy pulses through the first medical device to the tissue at the medical site to continue the ablation operation.

19. The method as in claim 11 further comprising:
delaying activation of the second medical device with respect to a time of transitioning from operating in the first mode to operating in the second mode.

20. A non-transitory computer-readable storage medium having instructions stored thereon, the instructions, when executed by computer processor hardware, cause the computer processor hardware to:
implement a control sequence that switches between different operational modes over time, the different operational modes including a first mode and a second mode;
enable a first medical device to output an energy signal during windows of time of operating in the first mode to perform a medical operation at the medical site; and
disable the first medical device from outputting the energy signal during windows of time of operating in the second mode.

21. The system as in claim 1, wherein the second medical device is operable to monitor attributes of tissue being ablated by the first medical device during the first mode.

22. The system as in claim 1, wherein the second medical device includes an electrode assembly and a flexible link, the electrode assembly operable to monitor tissue at the medical site; and
wherein the second medical device further includes integrated circuitry electrically coupled to the electrode assembly, the integrated circuitry disposed in the flexible link.
